(19) United States
(12) Patent Application Publication
(10) Pub. No.: US 2004/0014679 A1
(43) Pub. Date: Jan. 22, 2004

(54) Inhalation powder containing the CGRP antagonist BIBN4096 and process for the preparation thereof

(75) Inventors: Michael Trunk, Ingelheim (DE); Claudius Weiler, Ingelheim-Grosswinterheim (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co., KG, Ingelheim (DE)

(21) Appl. No.: 10/365,361

(22) Filed: Feb. 12, 2003

Related U.S. Application Data
(60) Provisional application No. 60/363,705, filed on Mar. 12, 2002.

(30) Foreign Application Priority Data
Feb. 20, 2002 (DE) 102 07 026.1

(51) Int. Cl. A61K 38/04; A61L 9/04; A61K 9/14; A61K 31/517

(52) U.S. Cl. 514/19; 424/46; 514/252.17

(57) ABSTRACT

The invention relates to an inhalation powder for treating migraine, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine [BIBN4096] of formula I as the active substance base in the form of spherically nanostructured microparticles, and a process for the manufacture thereof.

### RELATED APPLICATIONS

Benefit of U.S. Provisional Application Serial No. 60/363,705, filed on Mar. 12, 2002 is hereby claimed.

### FIELD OF THE INVENTION

The invention relates to an inhalation powder containing the CGRP antagonist -[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine [BIBN4096] of formula I in the form of spherically nanostructured microparticles which are stable in their amorphous state under normal conditions (T<50° C., relative humidity <75%) and a process for the manufacture thereof by which the thermodynamically stable or stabilised active substance can be processed in its amorphous state in a single step to form microparticles.

The spherically nanostructured microparticles according to the invention are suitable for the preparation of inhalation powders in which no other excipients or additives (carrier materials) are required in order to obtain a powder which can be handled on an industrial scale, which can be further processed directly and has excellent properties in terms of dispersibility and is sufficiently easy to process with regard to its cohesive properties. In another aspect the invention relates to the inhalation powders which may be obtained using the process according to the invention.

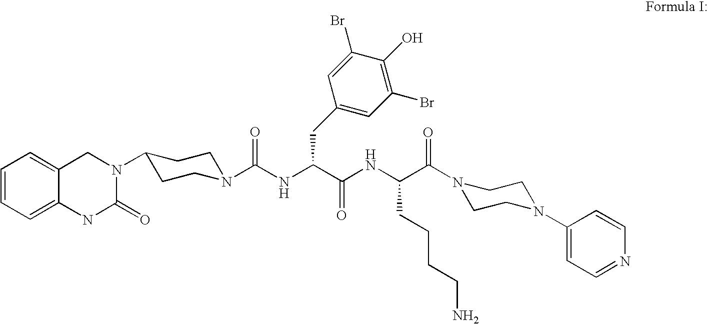

### PRIOR ART

BIBN4096 is a highly effective CGRP antagonist for the treatment of migraine, which is described in U.S. Pat. No. 6,344,449. It cannot be administered orally using conventional preparations as the substance has only limited bioavailability by oral route.

In the case of inhalation powders, inhalable powders which are packed into suitable capsules (inhalettes) are delivered into the lungs by means of powder inhalers. Alternatively, they may be inhaled by the use of suitable powdered inhalable aerosols which may contain, for example, an HFC134a, HFC227 or mixture thereof as propellant gas.

The microparticles of the pure active substance are administered through the airways to the surface of the lung, e.g. in the alveoli, by the inhalation process. These particles settle on the surface and can only be absorbed in the body after the dissolution process by active and passive transporting processes.

Inhalation systems are known in the literature wherein the active substance is present either as a micronised suspension in a suitable solvent system as the carrier, or in the form of a dry powder.

Usually, inhalation powders are prepared e.g. in the form of capsules for inhalation based on the general teaching as described in DE-A-179 22 07, using the chemically most stable form of the active substance. Pharmaceutical preparations prepared by mixing a finely divided medicament with a coarser carrier medium are dispersed in an air current by a so-called “powder flow method” using the suction mode of the inhaler as the main energy source.

A critical factor in multi-substance systems of this kind is the uniform distribution of the pharmaceutical composition in the powder mixture. Moreover, the carrier results in additional stress on the lungs as well as the occurrence of undesirable interactions, which may lead to problems of compatibility.

One significant aspect of the administration of the active substance by inhalation is that only particles of a specific aerodynamic size enter the target organ, namely the lungs. The particle size of these particles destined for the lungs (inhalable fraction) is in the submicron range. Such particles are conventionally produced by micronisation (grinding in an air stream). As a result, such particles may often be of complex composition in terms of their crystal properties as a result of this mechanical step. Similarly, the geometric form of the particles of starting material also determines the morphological properties of the micronised material.

Apart from the jet grinding process, the airstream grinding process being of particular significance, it is also possible to produce a suitable micronised product by alternative methods. Suitable micronising processes for preparing microparticles in the submicron range include, for example, the precipitation method including the processes in which the active substance can be precipitated as a non-crystalline (amorphous) solid by evaporating the solvent beyond its maximum solubility, precipitation by means of supercritical gases, such as the RESS or PGSS process (J. Jung, M. Perrut: Particle Design Using Supercritical Fluids, J. Supercrit. Fluids 20 (2001), 179-219), the GASR process (M. P. Gallager et al.: Gas Antisolvent Recrystallization, Am. Chem. Soc. (1989)), the PCA process (D. J. Dixon, K. P. Johnston: Polymeric Materials Formed by Precipitation with compressed Fluid Antisolvent, AIChE Journal (1993, Vol. 39(1), 127), freeze-drying, spray drying or a combination of several of the abovementioned processes.

It is known from the literature that lung-bound particles measuring between 0.5 μm and 10 μm, preferably between 0.5 μm and 6 μm, can be produced by spray-drying. Industrially usable formulations can normally be prepared from spray-dried particles of this kind using the method mentioned above (DE-A-179 22 07) which have sufficient dispersibility for medical use (inhalation) [Y.-F. Maa, P.-A. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P. A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109].

In addition to these examples there are other methods of production, proposed by pharmaceutical companies in particular, based on spray-drying processes, which describe special formulations for inhalation powders.

Apart from the requirements set out hereinbefore, it should generally be borne in mind that any change to the solid state of a pharmaceutical composition which can improve its physical and chemical stability as well as its technical qualities provides a considerable advantage over less stable forms of the same medicament.

### STATEMENT OF THE PROBLEM

The complex objective of the present invention was primarily to provide a bioavailable formulation for the highly effective CGRP antagonist BIBN4096. The formulation according to the invention should have a rapid onset of activity for the treatment of acute pain or, in the case of migraine, pain with a very sudden onset. This means that rapid absorption of the active substance and a fast rise in the plasma level must be ensured.

### DESCRIPTION OF THE INVENTION

A rapid onset of activity for the treatment of acute pain and for achieving a high plasma level of the salts of the active substance BIBN4096 within the shortest possible time can best be achieved, apart from by intravenous administration, via the lungs as the receiving organ.

Within the scope of the present invention it has now, surprisingly, been found that BIBN4096 in the form of the active substance base may be made sufficiently bioavailable by administering it by inhalation. It has been found that when the active substance is administered by inhalation in the form of spherically nanostructured microparticles a bioavailability of about 60% based on the fine fraction of the formulation (corresponding to FPD determined according to USP 24 Suppl. 2000) can be achieved.

The formulation according to the invention does not require the addition of any carrier materials.

A first object of the present invention is thus an inhalation powder containing the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine [BIBN4096] of formula I in the form of spherically nanostructured microparticles, characterised in that

(a) the particles have a specific surface area of between 1 m²/g and 25 m²/g, preferably between 1 m²/g and 20 m²/g, most preferably between 3 m²/g and 10 m²/g,

(b) the characteristic value Q(5.8) is between 50% and 100% and

(c) the parameter X₅₀ is between 1 μm and 6 μm.

These microparticles are characterised by special physical and physico-chemical properties which lead to improved pharmacological/pharmacokinetic properties when the substance is administered. The availability of the substance—both quantitative, based on the quantity of active substance administered, and also based on a high plasma level to be achieved as quickly as possible—is determined not only by the biochemical properties of the substance but also by physicochemical properties. If a solid is administered, as in the case of an inhalation powder, the parameters of absolute solubility in the ambient medium and also the speed of dissolution in the ambient medium as a function of the local concentration of the active substance and time should be taken into consideration in particular.

Optimum administration by inhalation must therefore take into account the fact that the particles of active substance form a finely divided coating over the surface of the lungs. The crucial factor here is that the active substance is changed in such a way that the microparticles to be inhaled have advantages in terms of their particle-to-particle interaction and their dispersion or aerodynamic properties which mean that on the one hand the particles are deposited quantitatively in the deeper parts of the lungs and on the other hand the maximum possible surface area of the lungs is covered. Therefore, the physical-chemical properties of the microparticles to be inhaled are of major importance in inhalation powders.

The particles produced according to the invention have high physical stability. In particular, the properties of the particles when used as an inhalation powder enable a high proportion of fine particles to be realised, technically determined, for example, by cascade impactor measurement (Andersen Cascade Impactor, according to USP 24 or Pharm. Eur. Suppl. 2000). Typically, the proportion of the particles according to this method which are less than 5 μm in size (aerodynamically) is greater than 15%, while in some cases fine fractions of more than 50% are achieved. Apart from this key parameter for inhalable substances, the powder is characterised in that it can be further processed by current technical processes. Powders produced in this way are characterised by the physicochemical parameters of particle size, e.g. measured by laser diffraction, as well as specific surface, e.g. measured by multipoint B.E.T. measurement. For the characteristic value Q^{(5.8)} the particle size of powders thus produced is typically between 50% and 100%, and for the parameter X₅₀ it is between 1 μm and 6 μm. Particles which are produced by the above methods typically have values for the specific surface of between 1 m²/g and 25 m²/g, ideally between 1 m²/g and 20 m²/g, most preferably between 3 m²/g and 10 m²/g. Geometrically, particles produced by the above methods have particle shapes which may be described, depending on the test conditions, between the extremes of “spherical shape”, “spherical shape with cavity, optionally with hole”, “spherical shape with inwardly shaped convexities”, as well as “collapsed hollow body”. Under the scanning electron microscope the surface of such particles is substantially nanostructured.

It has been found according to the invention that BIBN4096 in the form of the free base can surprisingly be changed morphologically by a spray drying process in such a way that a powder prepared in this way can be transferred directly into a primary packaging means without any further steps, specifically without the need to mix it with a coarser carrier material, and can be delivered from said packaging means for inhalation by means of a powder inhaler.

The manufacturing process may be controlled so that the particles are present in a suitable particle size, normally between 0.1 and 10 μm, and these particles have surface characteristics such that they are easy to fluidise/disperse.

It has also been found that the particle morphology including the particle size can be critically controlled by the choice of process parameters and manufacturing parameters. One surprising factor is that powders of this substance which have been micronised by “conventional” stream grinding processes and are present in a comparable particle size spectrum nevertheless differ fundamentally in their morphology from particles produced according to this invention, in terms of their surface characteristics/particle-to-particle interactions. This is apparent from the fact that the quality parameter known as the “Fine Particle Fraction of Delivered Dose” (e.g. according to the method of determining the “Aerodynamic Particle Size Distribution”—USP 24 or Pharm. Eur. Suppl. 2000) is improved by a factor 10 or more. As there is no need for a carrier material in the formulation either, the absolute dose of active substance actually available to the patient in a given total amount of powder administered is improved by a significantly higher factor.

The method of preparation according to the invention is characterised in that the active substance is suitably dissolved, sprayed and dried in a spray tower. The principle of spray-drying consists of breaking up a solution or suspension of the product which is to be dried into fine droplets and drying them with a hot gas current. The solid fraction remaining after the solvent has evaporated is separated off from the gas current by means of an inertia force separator (e.g. cyclone) and/or by a filter unit and collected. The microparticles thus produced are characterised by special values in terms of particle size, specific surface area and morphology.

Organic solvents or organic aqueous solvent mixtures have proved suitable as solvents. Preferably, an alcoholic aqueous solvent system is used, more preferably a solvent mixture consisting of ethanol/methanol/water and ethanol/propanol/water and most preferably the solvent mixture of ethanol and water. The molar proportion of water in the solvent mixtures should range from 0.1 to 10 times the amount of the molar proportion of the alcohol components, preferably from 0.5 to 4 times the amount.

The adjustment of the active substance concentration is intended primarily to make the process economical. However, limits are imposed on the active substance concentration which may be selected, these limits being set by the fact that the surface qualities of the particles can be optimised by a specific ratio between the droplet size and solids concentration. Normally, a concentration of between 0.5 and 20% by weight, preferably between 2 and 10 percent by weight, most preferably between 3 and 8 percent by weight should be selected. The droplet size is a critical parameter for the production of inhalable particles. Depending on the nozzle used the throughput of spray gas should be selected in conjunction with the throughput of solution so as to achieve the desired droplet size. As there are a number of combinations of the parameters “nozzle—throughput of spray gas—throughput of solution” which result in a suitable droplet size, the process can sensibly be defined by the droplet size which is to be selected for the process. This may be characterised by the characteristic value X₅₀ (median value=particle size/droplet size, below which 50% of the quantity of particles are found, with regard to the volume distribution of the individual particles/droplets), which should be in the range between 1.5 μm and 20 μm, preferably between 1.5 μm and 8 μm, as well as the characteristic value Q_{(5.8)} (corresponding to the quantity of particles below 5.8 μm, based on the distribution by volume of the particles), which should be between 10% and 100%, preferably between 30% and 100%.

On an industrial scale this is achieved by using a suitable commercial nozzle, e.g. single- or multi-substance nozzles which exhibit these characteristics as a function of the nozzle parameters (e.g. speed of rotation in the case of rotary atomisers or applied pressure and the resulting mass flow of the atomising gas in the case of two-substance nozzles) as well as the spray rate (volumetric flow of “spray solution”). Apart from the special conditions which have to be adhered to during the actual spraying process, in order to generate suitable droplets for the drying process, it is apparent that the surface characteristics of the particles may also be positively or deliberately influenced by the choice of the drying parameters. The critical characteristics which impinge on the drying step are the inlet and outlet temperature of the drying gas and the volumetric flow of the drying gas passed through. Care should be taken to ensure that the droplets of suitable size are passed through the drying chamber in such a way that the droplets and the dried particles do not come into contact, or only come into slight contact, with the wall of the spray tower. This is achieved by the use of nozzles with a corresponding spray cone, by a spray tower of suitable diameter and by the flow conditions in the apparatus. The starting temperature must be adapted to the process so that the powder has a sufficiently low residual solvent content and thus a sufficient chemical and physical stability is achieved. This is ideally obtained if the starting temperature is maintained in the region of the boiling temperature or slightly above. By contrast, the inlet temperature of the drying gas must be selected so that in conjunction with the parameter “volumetric flow of drying gas” and the spray rate, the drying is gentle enough to produce particles with suitable surface qualities.

A second object of the invention is thus a process for preparing the active substance base BIBN4096 in the form of spherically nanostructured microparticles, comprising the steps of

a) dissolving the active substance BIBN4096 in an organic solvent or an organic-aqueous solvent mixture to prepare a solution of the active substance with an active substance concentration of between 0.5 and 20 percent by weight, preferably between 2 and 10 percent by weight, most preferably between 3 and 8 percent by weight,

b) spraying the resulting solution of active substance in the usual way so as to obtain a spray mist with a droplet size having the characteristic value X₅₀ in the range from 1.5 to 20 μm, preferably from 1.5 to 8 μm, and Q_{(5.8)} between 10% and 100%, preferably between 30% and 100%,

c) drying the spray mist thus obtained by means of a drying gas, while applying the following parameters:

an inlet temperature of the drying gas of 100° C. to 350° C., preferably between 120° C. and 250° C. and more preferably between 130° C. and 200° C.,

an outlet temperature of the drying gas of 40° C. to 120° C.,

a volumetric flow of the spray gas of 1 Nm³/h to 15 Nm³/h,

a volumetric flow of the drying gas of 15 Nm³/h to 1500 Nm³/h, preferably 15 Nm³/h to 150 Nm³/h, and

d) separating the dried solid fraction from the drying gas current in conventional manner.

A third object of the invention is the use of the active substance base BIBN4096 in the form of spherically nanostructured microparticles which may be obtained by the process described above, for preparing an inhalation powder.

A fourth object of the present invention is an inhalation powder, characterized in that the spherically nanostructured microparticles are obtainable by the above-mentioned process according to the invention.

Experimental Section

1) Methods of Measurement

a) Determining the Particle Size by Laser Diffraction (Fraunhofer Diffraction):

| | |
|---|---|
| Method of | In order to determine the particle size, the powder is |
| measurement: | placed in a laser diffraction spectrometer by means of |
| | a dispersing unit. By the median value X₅₀ is meant |
| | the particle size below which 50% of the particles are |
| | found. The Q_{(5.8)} value describes the percentage |
| | proportion of the particles which are less than 5.8 μm |
| | in size. |
| Measuring | Laser diffraction spectrometer (HELOS), Messrs. |
| equipment: | Sympatec |
| Software: | WINDOX 4 Version 3.3/REL 1 for Examples 1 to 3 |
| | and Version 4 for Examples 4 to 6 |
| Dispersing unit: | RODOS/dispersing pressure: 3 bar |
| Focal length: | 100 mm [measuring range: 0.9 . . . 175 μm] |
| Evaluation mode: | HRLD (V 3.3 Rel. 1) |

b) Determining the Specific Surface:

| | |
|---|---|
| Method of | The specific surface is determined by exposing |
| measurement: | the powder sample to a nitrogen atmosphere at |
| | different pressures. Cooling the sample causes |
| | the nitrogen molecules to be condensed on the |
| | surface of the particles. The quantity of |
| | condensed nitrogen is determined by means of |
| | the pressure drop in the system and the specific |
| | surface of the sample is calculated by means of |
| | the surface nitrogen requirement and the weight |
| | of the sample. |
| Measuring | Tri Star Multi Point BET, Messrs. Micromeritics |
| equipment | |
| Heating station: | VacPrep 061, Messrs. Micromeritics |
| Heating: | about 12 h/40° C. |
| Analytical parameters | |
| Sample container: | ½ inch; with “filler rod” |
| Methods of analysis: | 16 point BET surface measurement |
| | 0.05 to 0.20 p/p0 |
| absolute pressure | 5.0 mm Hg |
| tolerance: | |
| relative pressure | 5.0% |
| tolerance: | |
| speed of evacuation: | 50.0 mm Hg/second |
| evacuation threshold: | 10.0 mm Hg |
| duration: | 0.1 h |
| void volume: | lowering of Dewar vessel, t: 0.5 h |
| retention time: | 20 seconds |
| Minimum equilibration | 600 seconds |
| time: | |
| Adsorbent: | nitrogen |

c) Determining the Droplet Size by Laser Diffraction (According to Mie):

| | |
|---|---|
| Measuring | Laser diffraction spectrometer (HELOS), Messrs. |
| equipment: | Sympatec |
| Software: | WINDOX Version 4 |
| Focal length: | 100 mm [Measuring range: 0.9 . . . 175 μm] |
| Method of | The droplet size is determined by removing the nozzle |
| measurement: | from the spray drier and placing the spray in the upper |
| | third of the spray cone centrally in the laser beam. The |
| | measurement is taken at ambient temperature with water |
| | as the reference medium under otherwise identical |
| | conditions. |

### 2) EXAMPLES OF SPRAY PARAMETERS

### Example 1

### Spray Parameter, Suitable for an Alcoholic BIBN4096 Solution (Modified BÜCHI Spray Drier)

| Concentration of solution/ | | 7 | g BIBN 4096 in 100 ml |
|---|---|---|---|
| composition solvent | | | ethanol/methanol/H₂O |
| | | | molar ratio: 1:1:2.3 |
| Droplet size | Q_{(5.8)} | 46% | (evaluated according to Mie); |
| (reference solution: | | 51% | (evaluated according to |
| H₂O at ambient | | | Fraunhofer) |
| temperature) | | 6.3 | μm (evaluated according to Mie); |
| | X₅₀ | 5.7 | μm (evaluated according to |
| | | | Fraunhofer) |

| | | |
|---|---|---|
| volumetric flow “spray rate” | 18 | ml/min |
| spray pressure (nozzle type) | 2.9 | bar overpressure (N₂) |
| | | (BÜCHI spray nozzle 0.7 mm, |
| | | Art. no. 04364) |
| volumetric flow “atomising | 1775 | standard litres/h |
| pressure” (nozzle type) | | (BÜCHI spray nozzle 0.7 mm, |
| | | Art. no. 04364) |
| entry temperature | 150° | C. |
| exit temperature | 100° | C. |
| volumetric flow “drying gas” | 30 | standard m³/h |
| cross section of drying tower | 105 | mm |

Characterisation of the Solid Particles Obtained

| | | | |
|---|---|---|---|
| particle size | X₅₀ | 1.9 | μm |
| | Q_{(5.8)} | 98.3% | |

### Example 2

### Spray Parameter, Suitable for an Alcoholic BIBN4096 Solution (Modified BÜCHI spray drier)

| Concentration of solution/ | | 7 | g BIBN 4096 in 100 ml |
|---|---|---|---|
| composition solvent | | | ethanol/methanol/H₂O |
| | | | molar ratio: 1:1:2.3 |
| Droplet size | Q_{(5.8)} | 27% | (evaluated according to Mie) |
| (reference solution: | | 39% | (evaluated according to |
| H₂O at ambient | | | Fraunhofer) |
| temperature) | | 8.9 | μm (evaluated according to Mie) |
| | X₅₀ | 7.3 | μm (evaluated according to |
| | | | Fraunhofer) |

| | | |
|---|---|---|
| volumetric flow “spray rate” | 18 | ml/min |
| spray pressure (nozzle type) | 2.9 | bar overpressure (N₂) |
| | | (BÜCHI spray nozzle 0.7 mm, |
| | | Art. no. 04364) |
| volumetric flow “atomising | 1482 | standard litres/h |
| pressure” (nozzle type) | | (BÜCHI spray nozzle 0.7 mm, |
| | | Art. no. 04364) |
| entry temperature | 150° | C. |
| exit temperature | 100° | C. |
| volumetric flow “drying gas” | 30 | standard m³/h |
| cross section of drying tower | 105 | mm |

Characterisation of the Solid Particles Obtained:

| | | | |
|---|---|---|---|
| particle size | X₅₀ | 2.4 | μm |
| | Q_{(5.8)} | 87% | |

### Example 3

### Spray Parameter, Suitable for an Alcoholic BIBN4096 Solution (Modified BÜCHI Spray Drier)

| Concentration of solution/ | | 7 | g BIBN 4096 in 100 g |
|---|---|---|---|
| composition solvent | | | ethanol/methanol/H₂O |
| | | | molar ratio: 1:1:2.3 |
| Droplet size | Q_{(5.8)} | <10% | |
| (reference solution: | X₅₀ | 17 | μm |
| H₂O at ambient | | | |
| temperature) | | | |

| | | |
|---|---|---|
| volumetric flow “spray rate” | 1.048 | l/h |
| spray pressure (nozzle type) | 0.8 | bar overpressure (N₂) |
| | | (BÜCHI spray nozzle 0.7 mm, |
| | | Art. no. 04364) |
| volumetric flow “atomising | 0.6 | kg/h (BÜCHI spray nozzle 0.7 |
| pressure” (nozzle type) | | mm, Art. no. 04364) |
| entry temperature | 150° | C. |
| exit temperature | 100° | C. |
| volumetric flow “drying gas” | 35-36 | standard m³/h |
| cross section of drying tower | 105 | mm |

Characterisation of the Solid Particles Obtained:

| | | | |
|---|---|---|---|
| particle size | X₅₀ | 5.7 | μm |
| | Q_{(5.8)} | 50.7% | |
| Specific surface | Sₘ | 19.6 | m²/g |

### Example 4

### Spray Parameter, Suitable for an Alcoholic BIBN4096 Solution (Modified BÜCHI Spray Drier)

| Concentration of solution/ | | 7.0 | g BIBN 4096 in 100 g |
|---|---|---|---|
| composition solvent | | | ethanol/methanol/H₂O |
| | | | molar ratio: 1:1:2.3 |
| Droplet size | Q_{(5.8)} | 59% | |
| (reference solution: | X₅₀ | 6.5 | μm |
| H₂O at ambient | | | |
| temperature) | | | |

| | | |
|---|---|---|
| volumetric flow “spray rate” | 1.08 | l/h |
| spray pressure (nozzle type) | 5.5 | bar overpressure (N₂) |
| | | (BÜCHI spray nozzle 0.7 mm, |
| | | Art. no. 04364) |
| volumetric flow “atomising | 3.4 | kg/h (BÜCHI spray nozzle 0.7 |
| pressure” (nozzle type) | | mm, Art. no. 04364) |
| entry temperature | 150° | C. |
| exit temperature | 95° | C. |
| volumetric flow “drying gas” | 36 | standard m³/h |
| cross section of drying tower | 105 | mm |

Characterisation of the Solid Particles Obtained:

| | | |
|---|---|---|
| particle size | X₅₀ | 1.4 μm |
| | Q_{(5.8)} | 99.7% |
| Specific surface | Sₘ | 7.3 m²/g |

### Example 5

### Spray Parameter, Suitable for an Alcoholic BIBN4096 Solution (Modified BÜCHI Spray Drier)

| Concentration of solution/ | | 9.9 g BIBN 4096 in 100 g ethanol/ |
|---|---|---|
| composition of solvent | | H₂O molar ratio: 2:3 |
| Droplet size | Q_{(5.8)} | 59% |
| (reference solution: H₂O | | |
| at ambient temperature) | | |
| | X₅₀ | 6.5 μm |
| volumetric flow “spray rate” | | 1.2 l/h |
| spray pressure (nozzle type) | | 5.4 bar overpressure (N₂) |
| | | (BÜCHI spray nozzle 0.7 mm, |
| | | Art. no. 04364) |
| volumetric flow “atomising | | 3.4 kg/h |
| pressure” | | (BÜCHI spray nozzle 0.7 mm, |
| (nozzle type) | | Art. no. 04364) |
| entry temperature | | 150° C. |
| exit temperature | | 101° C. |
| volumetric flow “drying gas” | | 36 standard m³/h |
| cross section of drying tower | | 105 mm |

Characterisation of the Solid Particles Obtained:

| | | |
|---|---|---|
| particle size | X₅₀ | 2.7 μm |
| | Q_{(5.8)} | 90.0% |
| Specific surface | Sₘ | 5.7 m²/g |

### Example 6

### Spray Parameter, Suitable for an Alcoholic BIBN4096 Solution (Modified BÜCHI Spray Drier)

| Concentration of solution/ | | 4.0 g BIBN 4096 in 100 g ethanol/ |
|---|---|---|
| composition of solvent | | H₂O molar ratio: 2:3 |
| Droplet size | Q_{(5.8)} | 59% |
| (reference solution: H₂O | | |
| at ambient temperature) | | |
| | X₅₀ | 6.5 μm |
| volumetric flow “spray rate” | | 1.2 l/h |
| spray pressure (nozzle type) | | 5.5 bar overpressure (N₂) |
| | | (BÜCHI spray nozzle 0.7 mm, |
| | | Art. no. 04364) |
| volumetric flow “atomising | | 3.4 kg/h |
| pressure” | | (BÜCHI spray nozzle 0.7 mm, |
| (nozzle type) | | Art. no. 04364) |
| entry temperature | | 150° C. |
| exit temperature | | 100° C. |
| volumetric flow “drying gas” | | 36 standard m³/h |
| cross section of drying tower | | 105 mm |

Characterisation of the Solid Particles Obtained:

| | | |
|---|---|---|
| particle size | X₅₀ | 1.5 μm |
| | Q_{(5.8)} | 99.7% |
| Specific surface | Sₘ | 7.5 m²/g |

Brief Description of the Figures

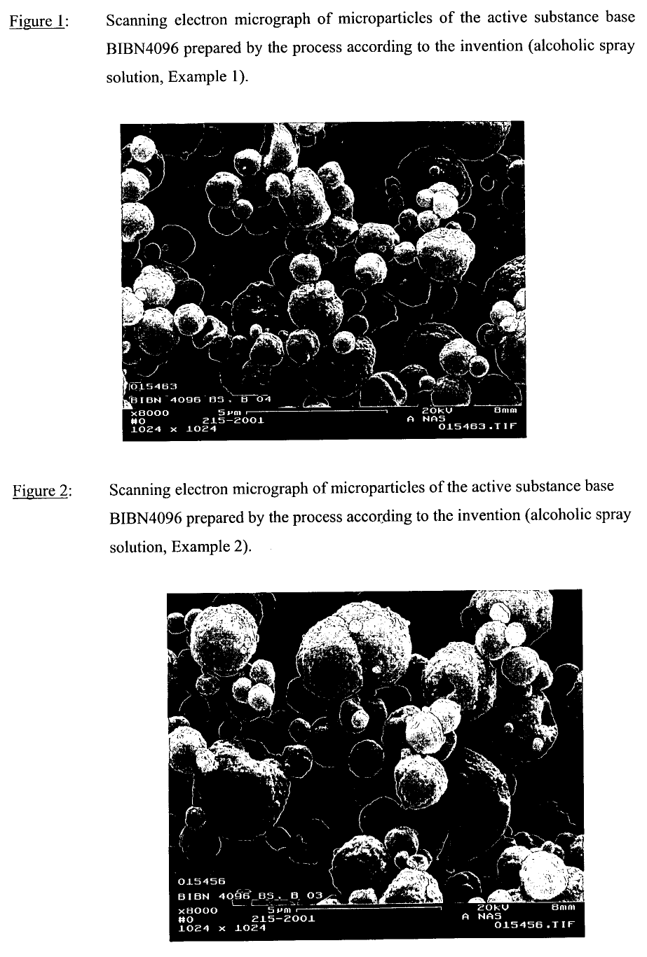

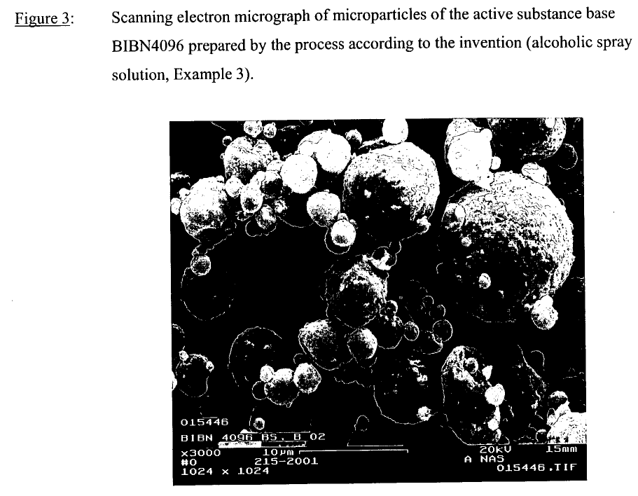

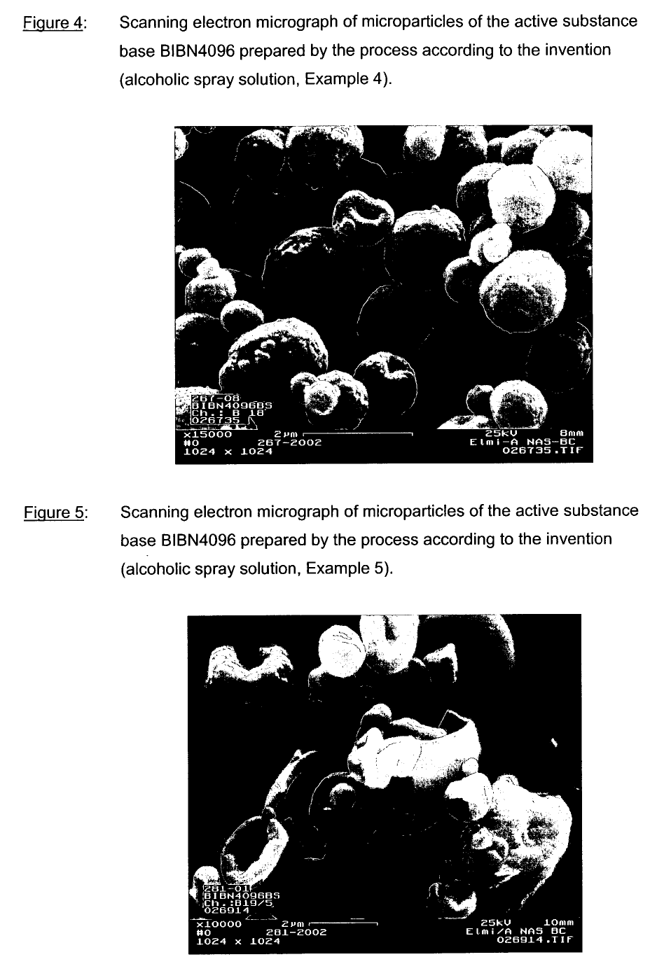

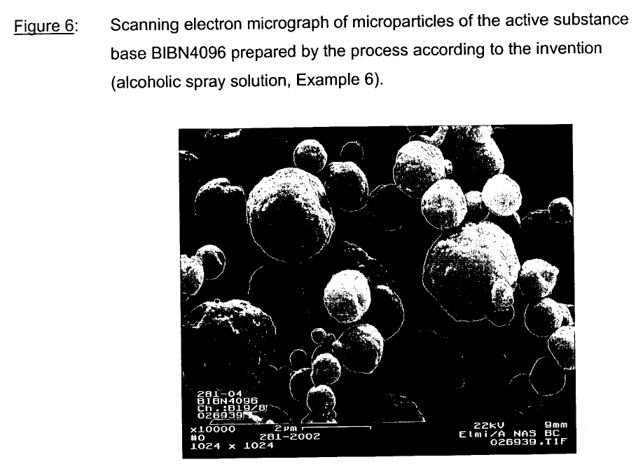

FIGS. 1 to 6 show photographs of microparticles of the active substance base BIBN4096 prepared from an alcoholic spray solution by the method according to the invention.

1. An inhalation powder containing the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine [BIBN4096] of formula 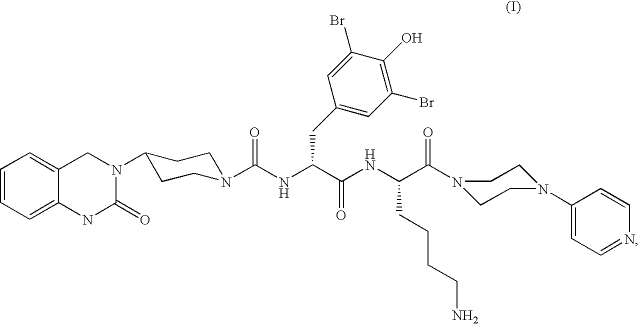
in the form of spherically nanostructured particles, wherein:
(a) the particles have a specific surface area of between 1 m²/g and 25 m²/g,
(b) the characteristic value Q_{(5.8)} is between 50% and 100% and
(c) the parameter X₅₀ is between 1 μm and 6 μm.

2. An inhalation powder according to claim 1, wherein the particles have a specific surface of between 1 m²/g and 20 m²/g.

3. An inhalation powder according to claim 1, wherein the particles have a specific surface of between 3 m²/g and 10 m²/g.

4. A process for preparing the active substance base BIBN4096 in the form of spherically nanostructured microparticles, comprising the steps of:
a) dissolving the active substance BIBN4096 in an organic solvent or an organic-aqueous solvent mixture to prepare a solution of the active substance with an active substance concentration of between 0.5 and 20 percent by weight,
b) spraying the resulting solution of active substance in conventional manner so as to obtain a spray mist with a droplet size having the characteristic value X₅₀ in the range from 1.5 to 20 μm,
c) drying the spray mist thus obtained by means of a drying gas, while applying the following parameters:
(1) an inlet temperature of the drying gas of 100° C. to 350° C.,
(2) an outlet temperature of the drying gas of 40° C. to 120° C.,
(3) a volumetric flow of the spray gas of 1 Nm³/h to 15 Nm³/h, and
(4) a volumetric flow of the drying gas of 15 Nm³/h to 1500 Nm³/h, and
d) separating the dried solid fraction from the drying gas current.

5. The process according to claim 4, wherein the solvent used to dissolve the active substance is an organic aqueous solvent system wherein the molar proportion of water to be used is from 0.1 to 10 times the molar proportion of the alcohol components.

6. The process according to claim 4, wherein the organic-aqueous solvent system consists of ethanol/methanol/water, wherein the molar proportion of water to be used is from 0.1 to 10 times the molar proportion of the alcohol components.

7. The process according to claim 4, wherein the organic-aqueous solvent system consists of ethanol/propanol/water, and wherein the molar proportion of water to be used is from 0.1 to 10 times the molar proportion of the alcohol components.

8. The process according to claim 4, wherein the organic-aqueous solvent system consists of ethanol/water, wherein the molar proportion of water to be used is from 0.1 to 10 times the molar proportion of the alcohol component.

9. The process according to claim 4, wherein the solution of the active substance used for spray-drying has a concentration of 2 to 10 percent by weight.

10. The process according to claim 4, wherein the solution of the active substance used for spray-drying has a concentration of 3 to 8 percent by weight.
